(19) United States
(12) Patent Application Publication
(10) Pub. No.: US 2016/0271365 A1
(43) Pub. Date: Sep. 22, 2016

(54) A DEVICE FOR ECG DERIVATION FROM A CATHETER

(71) Applicant: B. BRAUN MEDICAL SAS, Boulogne Billancourt (FR)

(72) Inventors: Simon John Forber, Ligugé (FR); Sylvain Massé, La Ciotat (FR)

(21) Appl. No.: 15/036,602

(22) PCT Filed: Nov. 17, 2014
(86) PCT No.: PCT/EP2014/074794

§ 371 Date: May 13, 2016

(30) Foreign Application Priority Data
Nov. 22, 2013 (FR) 1302710

(51) Int. Cl. A61M 25/01; A61B 5/00; A61M 25/09; A61B 5/042

(52) U.S. Cl. CPC A61M 25/0105; A61B 5/042; A61B 5/7278; A61M 25/09; A61M 2025/0166

(57) ABSTRACT

A device for the ECG derivation from a catheter can be used with a guide wire as well as without a guide wire, and can be manipulated in a simple and sterile manner. The device includes a pipe section exhibiting a channel that includes a contact pin which is connected to a terminal at the outer face of the device and is movable between a first position and a second position, the two positions representing different positions with respect to the channel axis.

### RELATED APPLICATIONS

This application is the U.S. national phase entry under 35 U.S.C. §371 of International Application No. PCT/EP2014/074794, filed Nov. 17, 2014, which is related to and claims the benefit of priority of French Application No. 1302710, filed Nov. 22, 2013. The contents of International Application No. PCT/EP2014/074794 and French Application No. 1302710 are incorporated by reference herein in their entirety.

### FIELD

The present invention relates to a device for ECG derivation from a catheter.

### BACKGROUND

In order to control the position of a catheter during its placement precisely, especially a central venous catheter, the catheter is displaced toward the heart after having punctured the vein under permanent ECG control until the potentials of the cardiac atrium appear on the screen. It would be dangerous to push the tip of the catheter further forward since it could reach the ventricle and cause arrhythmias. The catheter is then retracted by about 2 or 3 cm. By doing so, the atrium-specific potentials disappear, and the user knows that the tip of the catheter is now in front of the atrium which corresponds to the correct position of a central venous catheter.

To realize an intracardiac ECG, an electrically conducting connection needs to be established by means of a cardiac catheter for deriving the necessary signals.

The electrically conducting connection may be established in two different ways, namely by means of an electrically conductive guide wire or else by means of an electrically conductive liquid, in particular a saline solution which is introduced into the catheter.

Both methods may be necessary one after the other at different moments of a patient's treatment. Thus, it can prove to be advantageous to establish the electrical connection during the positioning of the catheter by means of a guide wire which is systematically used during the insertion of the catheter. In contrast, the utilisation of a physiological saline solution is advantageous during the subsequent position control so that a guide wire is not required to be reinserted into the catheter.

To allow ECG signals to be derived by means of an electrically conductive liquid, a device is known for example from document EP 0 153 952 B1 which is fixed at the free end of a catheter and allows an electrically conductive liquid to be introduced into the catheter by means of a syringe. An electrical contact, from which a connection cable to the ECG device is routed, is situated at the attachment piece of the syringe.

Document DE 43 18 963 C1 discloses a similar device which also allows the contact between a contact pin in a lateral attachment piece at the device and a guide wire to be established through an electrically conductive liquid, while the guide wire passes within a channel in the device.

The known solutions presuppose in any case the use of an electrically conductive liquid supplied from outside. The equipment used must be sterile and the amount of supplied liquid must be dosed with precision so as to ensure the electrical contact.

### SUMMARY

The object of the present invention is to propose a device for the ECG derivation from a catheter, which can be used with a guide wire as well as without a guide wire, and which can be manipulated in a simple and sterile manner.

The object of the invention is achieved by a device for the ECG derivation from a catheter, comprising a pipe section exhibiting a channel, which is characterized in that the channel comprises a contact pin which is connected to a terminal on the outer face of the device and is movable between a first position and a second position, the two positions representing different positions with respect to the channel axis.

The pipe section of such a device can be slipped onto a guide wire situated in a catheter for deriving a signal. The contact pin which is disposed within the channel of the pipe section is then in a first position, in which the passage of the guide wire through the pipe section is perfectly possible.

The contact pin can then be displaced to a second, different position with respect to the axis of the channel. In this position, the guide pin establishes an electrical connection with the guide wire. In this position of the guide pin, the guide wire can then be slightly trapped so that the device can no longer be freely displaced along the guide wire. An ECG device can be connected at the terminal on the outer side of the device which is connected to the contact pin. The signals from the tip of the guide wire are then transmitted to the ECG device without any loss.

The introduction of a conductive liquid into the catheter, which must be performed in a sterile manner, is not necessary in this case of application. The device may be located apart from the free end of the catheter on the guide wire and thus does not pose any sterility problem of the catheter.

The displacement of the contact pin may be linear or follow a circular trajectory. The device may be provided with a drum for instance, which is mounted to be rotatable about an axis perpendicular to the axis of the pipe section. The contact pin is disposed on the drum in an eccentric manner. The pin can move along a circular trajectory due to the rotation of the drum. The guide wire can introduce itself easily into the pipe section, provided the contact pin remains outside the axis of the pipe section. The rotation of the drum allows the contact pin to be set into a different position with respect to the axis of the tube section. Preferably, the trajectory of the pin crosses the axis of the pipe piece so that the guide wire is slightly under constraint to slightly leave the axis, and can thus be locked.

For example, the first and the second positions of the contact pin can be homothetic with respect to the axis of the pipe section. The contact can be established by rotating the drum by 180°.

In a preferred embodiment, the ECG derivation device exhibits at an end of the pipe section a terminal for a catheter. The device can also be used without a guide wire when an electrically conductive liquid, such as a physiological saline solution for example, is poured into the pipe section. The liquid then establishes the contact between the catheter and the contact pin, and the signal can be derived. A terminal for a syringe, in particular a female Luer lock, can be provided for introducing the liquid.

### BRIEF DESCRIPTION OF THE DRAWING FIGURES

Hereinafter, different embodiments of the invention are described in more detail by means of the annexed Figures, in which:

FIG. 1 is a schematic representation of the principle of the present invention;

FIG. 2a shows the schematic representation of a preferred embodiment of the present invention with the contact pin in the first position;

FIG. 2b shows the schematic representation of the embodiment of FIG. 2a with the contact pin in the second position;

FIG. 3a shows another preferred embodiment of the present invention in a sectional view;

FIG. 3b shows the use of the embodiment of FIG. 3a with the use of a guide wire; and

FIG. 3c shows the use of the embodiment of FIG. 3a without a guide wire.

### DETAILED DESCRIPTION

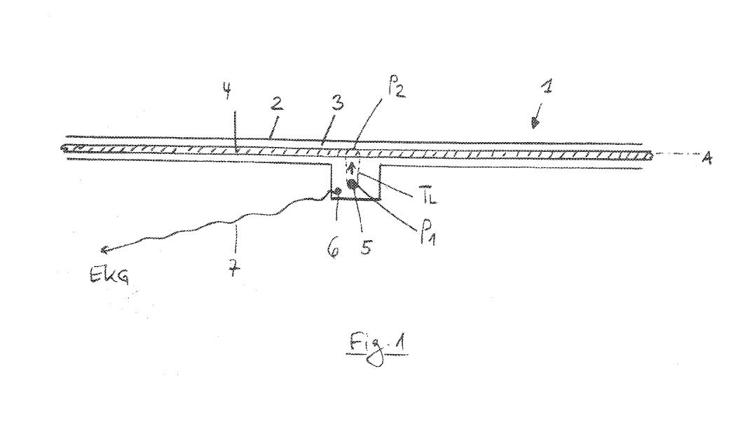

FIG. 1 is a schematic representation of the principle of the present invention. The Figure represents the ECG derivation device according to the invention with a pipe section 2 as a main part. The pipe section comprises a channel 3 inside thereof and is dimensioned to receive a guide wire 4. The inside of the channel 3 comprises a contact pin 5 which can move between a first position P₁ and a second position P₂ along the trajectory T_{L}, which is represented as a dotted line in the Figure.

The contact pin 5 is connected to a terminal/connection/access 6 disposed on the outer face of the device 1 by which a connection cable 7 can be branched off for connecting to an ECG device.

In the first position P₁, the contact pin is outside the axis A of the pipe section 2, so that the guide wire 4 can be easily introduced. The device can thus be easily displaced at the end of the guide wire 4 mounted in a catheter.

If the guide wire 4 is introduced into the channel 3 of the pipe section 2, the contact pin 5 can be displaced along its trajectory T_{L}. At this time, the pin enters the axis of the channel and forces the guide wire 4 slightly against the opposite wall of the channel 3 to establish a secure electrical contact between the contact pin 5 and the guide wire 4. Thus, an electrical connection between the end of the guide wire 4 situated within the patient, by means of the contact pin 5, the terminal 6 and the connection cable 7 is achieved with the ECG device not shown in this Figure.

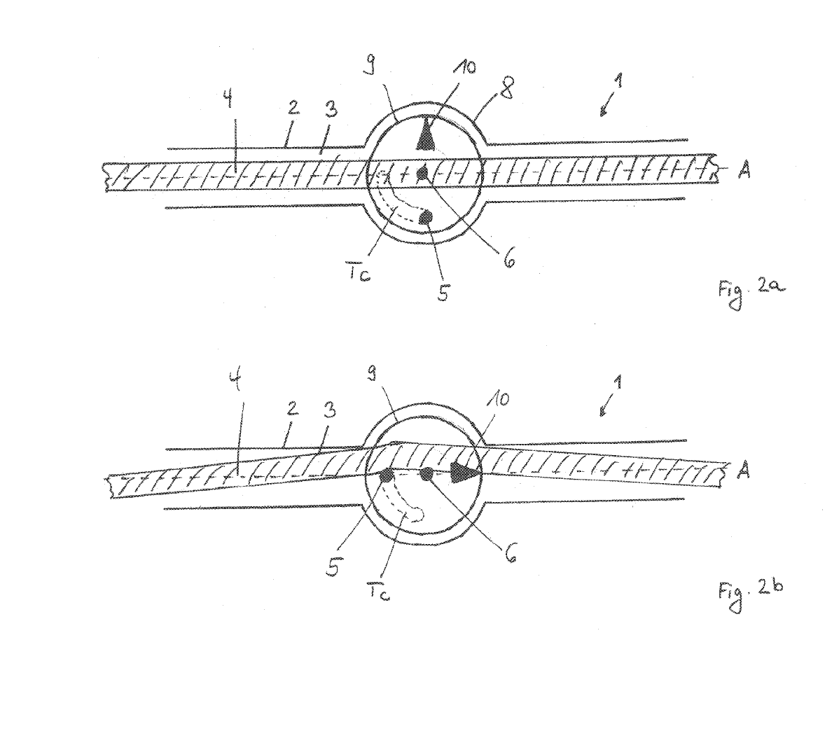

FIG. 2a shows the schematic representation of a preferred embodiment of the present invention with the contact pin 5 in the first position. The guide wire 4 is introduced into the channel 3 of the device 1 along the axis A.

The contact pin 5 is situated on a drum 9 rotatably mounted in a cylindrical extension 8 of the pipe section and is connected in an electrically conductive manner with the terminal 6 in the rotation axis of the drum. In the first position, the contact pin is situated outside the axis A of the pipe section without any contact with the guide wire 4.

FIG. 2b shows the schematic representation of the embodiment of FIG. 2a with the contact pin 5 in the second position. To this end, the drum 9 is rotated by about 90° using the handle 10 situated outside the device 1. The contact pin 5 moves along the circular trajectory T_{C} in the axis A of the channel 3 of the pipe section 2, and there abuts against the guide wire 4. The electrical contact is established. The position of the handle 10 permits to determine immediately whether the contact is established or not.

The device 1 can also be dimensioned such that the drum can be rotated by 180°, while the contact pin is arranged in the second position with respect to the channel axis in a homothetic manner relative the first position. In the second position, the contact pin 5 compresses the guide wire 5 again to withdraw it from its position along the axis A of the channel 3, and establishes an electrical contact. The advantage of this position is that the drum is rotatable by 360°, and even when it is forced, there is no risk of damaging pieces such as the contact pin inside the device.

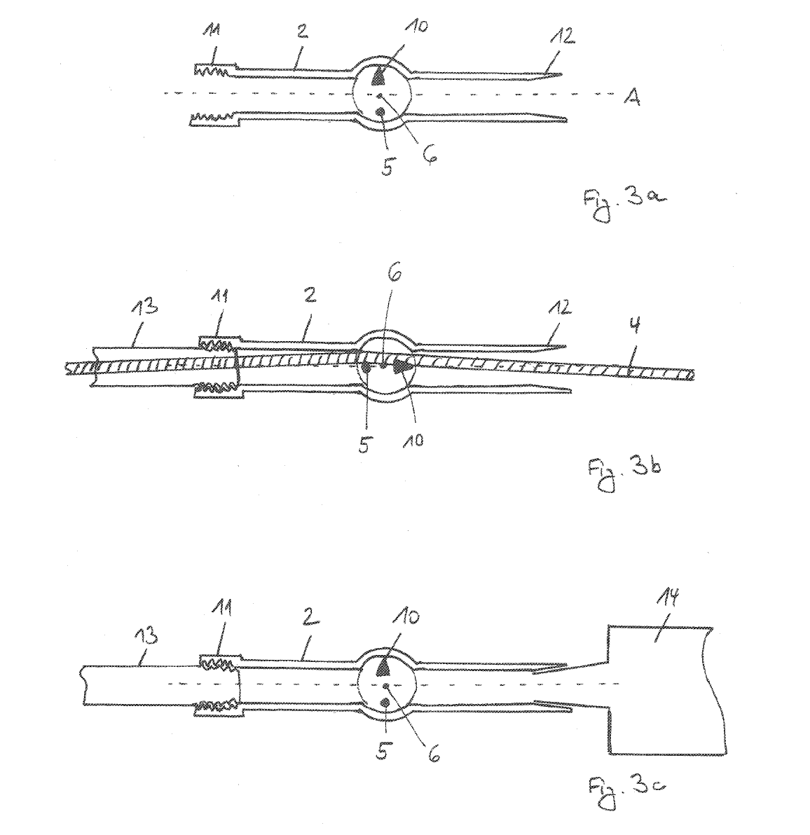

FIG. 3a shows another preferred embodiment of the present invention. Here, a terminal/connection/access 11 for a catheter is provided at an end of the pipe section 2, while a female Luer lock 12 is provided to connect to a syringe.

This embodiment of the invention is particularly advantageous in that it can be used both with a guide wire and an electrically conductive liquid. A catheter can be positioned first with the device, for instance, by means of a guide wire. For doing this, a contact can be established with a guide wired used during the positioning of the catheter. The introduction of a liquid, which is subjected to high requirements regarding sterility, is not necessary. The guide wire can be withdrawn once the catheter is positioned. The regular control of the catheter's position can be performed on the basis of the conventional liquid process without any need to use a separate device. On the one hand, there is no sterility problem as could have been the case when another device after the implantation of the catheter would have been added to its free end, on the other, savings can be realized in that the same single device is used both for the positioning of the catheter by means of a guide wire and subsequently without a guide wire.

FIG. 3b shows the device according to FIG. 3a in the case of usage with a guide wire 4. The terminal 11 is associated with a catheter 13 in which the guide wire 4 is situated. The guide wire 4 passes through the channel 3 of the device 1 and exits the channel 3 at the opposite end. The contact pin 5 is in the second position where it establishes an electrical contact with the guide wire 4.

FIG. 3c shows the device of FIG. 3b after the guide wire has been withdrawn. For doing this, the contact pin 5 is set in the first position which allows the guide wire to be withdrawn without any resistance. To ensure ECG signals to be derived, a commercially available syringe 14 including a sterile physiological saline solution is placed at the Luer lock 12 at the opposite end of the device 1, and the saline solution is introduced into the channel 3 of the device and thus into the catheter 13. The saline solution establishes an electrical contact with the contact pin 5 in the channel 3 of the device. The saline solution in the catheter 13 finally serves the purpose of establishing an electrical contact between the distal end of the catheter and the contact pin 5 and then by means of the terminal 6 and a suitable cable to the ECG device.

1. A device for ECG derivation from a catheter, comprising a pipe section exhibiting a channel,
the channel comprising a contact pin which is connected to a terminal at the outer face of the device and is movable between a first position and a second position, the two positions representing different positions with respect to the channel axis,
the channel further comprising a drum being arranged to be rotatable about an axis perpendicular to the channel axis, wherein the contact pin is arranged in an eccentric manner on the drum and, when the drum is rotated about its axis, the contact pin moves along a circular trajectory.

2. (canceled)

3. (canceled)

4. The device according to claim 1, wherein the contact pin crosses the axis of the channel during its transition from the first position to the second position.

5. The device according to claim 1, wherein the first position and the second position are homothetic with respect to the channel axis.

6. The device according to claim 1, wherein at one end of the pipe section, the device exhibits a terminal for a catheter.

7. The device according to claim 1, wherein at one end of the pipe section, the device exhibits a terminal for a syringe.
